(19) United States
(12) Patent Application Publication
(10) Pub. No.: US 2008/0306492 A1
(43) Pub. Date: Dec. 11, 2008

(54) CLIPPING INSTRUMENT FOR AN ENDOSCOPIC SURGICAL DEVICE

(75) Inventors: Hiroaki SHIBATA, Saitama (JP); Tomohiro KAWANO, Tokyo (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/123,694

(22) Filed: May 20, 2008

(30) Foreign Application Priority Data
Jun. 8, 2007 (JP) 2007-152343

(51) Int. Cl. A61B 17/10

(52) U.S. Cl. 606/143; 606/142

(57) ABSTRACT

A clipping instrument for an endoscope is provided. The clipping instrument includes a flexible sheath, an operation wire, which can be forwarded and retracted in an axial direction of the flexible sheath, at least one detachable clip assembly, including a pair of openable and closable clipping arms which can be projected forward to open outside the flexible sheath and to be closed to clip onto a treatment object outside the flexible sheath according to forwarding and retracting movements of the operation wire, and frictional members, which can generate frictional resistance between each other inside the flexible sheath. The frictional resistance is generated while the clipping arms of the clip assembly are forwarded by the forwarding movement of the operation wire to project out of the flexible sheath to be fully opened.

### BACKGROUND OF THE INVENTION

The present invention relates to a surgical device having a clipping instrument to be inserted through a forceps channel of an endoscope.

Generally, in a clipping instrument for endoscopic surgeries, specifically in a sequencing clip instrument with a plurality of detachable clips being aligned in line inside a flexible sheath, the clips are configured such that one of the clips at a distal end opens and closes as an operation wire, arranged in an axial direction of the flexible sheath, is advanced toward the distal end and retracted toward a proximal end. The clip at the distal end can be thus detached from a succeeding clip with its arms closed as the operation wire is further advanced forward and the succeeding clip opens. Such a configuration of the clipping instrument is disclosed in Japanese Patent Provisional Publication No. 2006-87537, for example.

When the conventional clipping instrument disclosed in the above-referenced publication is in use, operations of the clipping instrument, of which clips are opened/closed and advanced/retracted, are observed in images obtained through a display unit for the endoscope.

In such observation through the display unit, the clip being advanced is viewed from the rear portion; therefore, an operator may not be able to accurately recognize a posture and an attitude of the clip such as whether the clip is in a fully-open state, or amount of the clip being protruded from the sheath. The difficulty may cause an erroneous operation particularly when substantial clearness of the images is not ensured.

### SUMMARY OF THE INVENTION

In view of the foregoing drawback, the present invention is advantageous in that an endoscopic surgical device with a clipping instrument, in which conditions of the clips can be accurately recognized so that smooth operations can be conducted, is provided.

According to an aspect of the present invention, there is provided a clipping instrument for an endoscope. The clipping instrument includes a flexible sheath to be inserted into an instrument channel of the endoscope, an operation wire, which is configured to be forwarded and retracted in an axial direction of the flexible sheath, at least one detachable clip assembly, including a pair of openable and closable clipping arms. The at least one detachable clip assembly is arranged in vicinity of a distal end portion of the flexible sheath with the clipping arms closed. The clipping arms are configured to be projected forward to open outside the flexible sheath and to be closed to clip onto a treatment object outside the flexible sheath according to forwarding and retracting movements of the operation wire. The clipping instruments further include frictional members, which are configured to generate frictional resistance between each other inside the flexible sheath. The frictional resistance is generated while the clipping arms of the clip assembly in the vicinity of the distal end portion of the flexible sheath are forwarded by the forwarding movement of the operation wire to project out of the flexible sheath to be fully opened.

### BRIEF DESCRIPTION OF THE DRAWINGS

FIG. 1 is a cross-sectional side view of a clipping instrument for an endoscope according to a first embodiment of the present invention.

FIG. 2 is an exploded perspective view of a clip assembly of the clipping instrument according to the first embodiment of the present invention.

FIG. 3 is a side view of the clip assembly of the clipping instrument according to the first embodiment of the present invention.

FIG. 4 is a side view of the clip assembly of the clipping instrument with arms being opened according to the first embodiment of the present invention.

FIG. 5 is a side view of the clip assembly of the clipping instrument with arms being closed according to the first embodiment of the present invention.

FIG. 6 is an enlarged cross-sectional side view of the flexible sheath with a frictional ring and frictional cylinders according to the first embodiment of the present invention.

FIG. 7 is an overall cross-sectional side view of the clipping instrument in operation according to the first embodiment of the present invention.

FIG. 8 is an overall cross-sectional side view of the clipping instrument in operation according to the first embodiment of the present invention.

FIG. 9 is an overall cross-sectional side view of the clipping instrument in operation according to the first embodiment of the present invention.

FIG. 10 is an overall cross-sectional side view of the clipping instrument in operation according to the first embodiment of the present invention.

FIG. 11 is an overall cross-sectional side view of the clipping instrument in operation according to the first embodiment of the present invention.

FIG. 12 illustrates a frictional ring and the frictional cylinders according to a second embodiment of the present invention.

FIG. 13 illustrates the frictional rings and the frictional cylinder according to a third embodiment of the present invention.

FIG. 14 is an enlarged view of a frictional portion and a frictional cylinder according to a fourth embodiment of the present invention.

### DETAILED DESCRIPTION OF THE EMBODIMENT

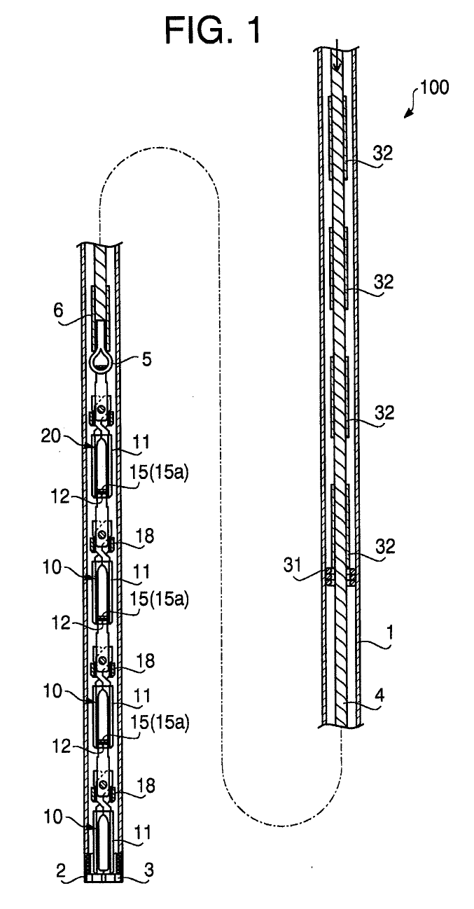

Hereinafter, referring to the accompanying drawings, an illustrative embodiment of the invention will be described. FIG. 1 is a cross-sectional side view of a clipping instrument 100 for an endoscope (not shown) according to a first embodiment of the present invention. The clipping instrument 100 includes a tubular flexible sheath 1. The flexible sheath 1 is made of, for example, poly-tetra-fluoro-ethylene and is inserted into a forceps channel (not shown) of the endoscope.

The flexible sheath 1 is provided with a sleeve 2 at a distal end, and a resilient C-shaped ring (C-ring) 3 made of a metal is arranged inside the sleeve 2. The C-ring 3 is restricted from moving in an axial direction of the flexible sheath 1, but can be resiliently expanded radially within the sleeve 2.

Inside the flexible sheath 1, an operating wire 4 to be advanced and retracted in parallel with the axial direction of the flexible sheath 1 is provided. The operating wire 4 is connected to an operating unit (not shown) at a proximal end thereof and can be advanced and retracted in accordance with an operation given to the operating unit. The operating wire 4 is fixedly connected to a connecting loop 5 through a connecting pipe 6.

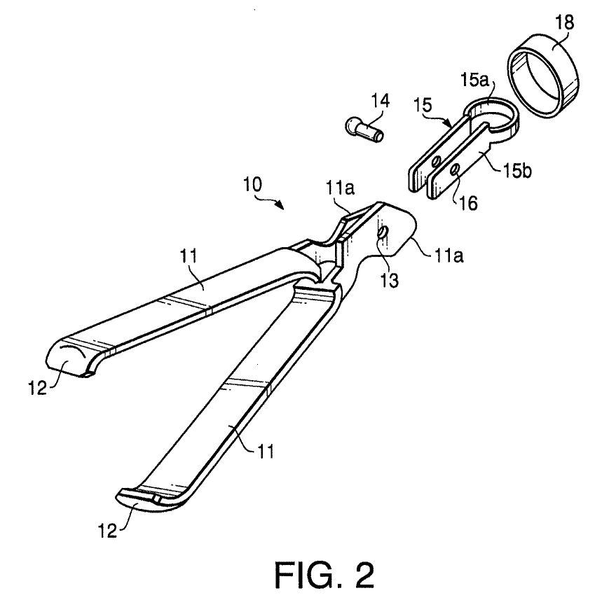

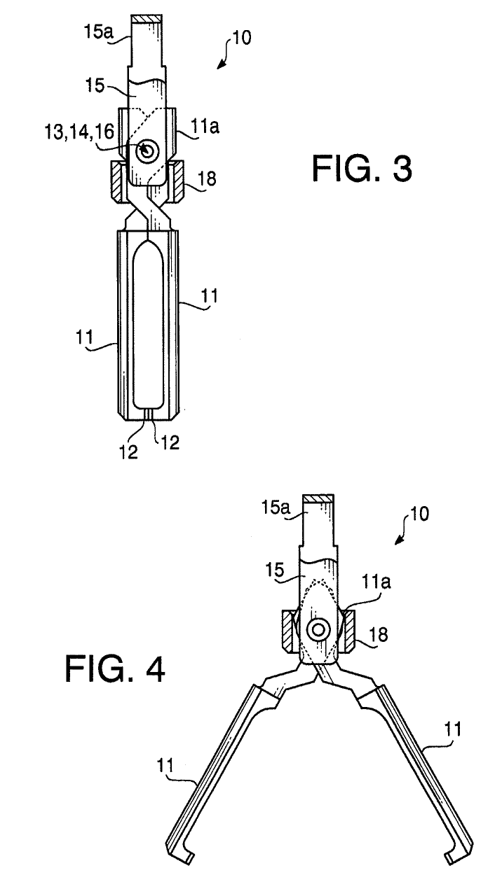

Further inside the flexible sheath 1, in vicinity to a distal end portion thereof, a plurality of clip assemblies 10 are aligned in line. FIG. 2 is an exploded perspective view of the clip assembly 10 of the clipping instrument 100 according to the embodiment of the present invention. FIG. 3 is a side view of the clip assembly 10 of the clipping instrument 100 according to the embodiment of the present invention. The clip assembly 10 is provided with a pair of arms 11, each of which is individually formed and provided with a claw portion 12. The claw portion 12 is curved inward, when the arms 11 are assembled, so that the two claw portions 12 are opposed to each other to clutch a treatment object.

Each of the arms 11 is formed to have a through hole 13 in vicinity to a proximal end thereof. A shaft 14 supporting the arms 11 is penetrated through the through holes 13 of the arms 11 so that the arms 11, rotated about the shaft 14, can be arbitrarily opened and closed.

The clip assembly 10 is further provided with a tail portion 15, which includes an open-ringed connecting loop 15a at a proximal end thereof, a pair of plate portions 15b, which are parallel to each other and loosely sandwich the proximal end portion of the arms 11 therebetween, and through holes 16 through which the shaft 14 is penetrated. The tail portion 15 is connected to the operating wire 4 through another clip assembly 10 being aligned at a proximal side and a connecting clip 20, which will be described later.

Furthermore, the clip assembly 10 includes a fastening ring 18, by which the arms 11 are forcibly closed. When the arms 11 are allowed to be open, the fastening ring 18 is loosely fit around the proximal portion of the arms 11, as shown in FIG. 3.

FIG. 4 is a side view of the clip assembly 10 of the clipping instrument 100 being opened according to the embodiment of the present invention. When the fastening ring 18 is retracted rearward to an opening position shown in FIG. 4, in which the fastening ring 18 becomes in contact with plate portions 11a of the arms 11. Thus, the arms 11 are opened.

When one of the clip assemblies 10 is projected out of the flexible sheath 1, the fastening ring 18 becomes in contact with the C-ring 3 at the distal portion thereof and is stopped thereat; therefore, the arms 11 can be opened. If the clip assembly 10 is forwarded further with more pressure, the fastening ring 18 outstretches the C-ring 3 and is projected forward from the sleeve 2 through the C-ring 3.

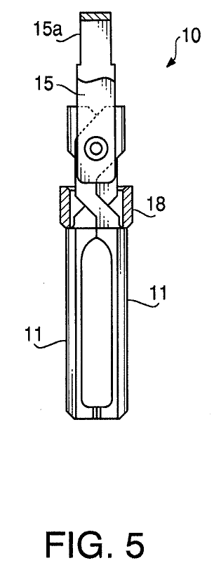

In reverse, when the fastening ring 18 is forwarded, as shown in FIG. 5, the arms 11 are forcibly closed. FIG. 5 is a side view of the clip assembly 10 of the clipping instrument 100 with arms 11 being closed according to the embodiment of the present invention. When the tail portion 15 is retracted inward the flexible sheath 1 after the fastening ring 18 is once projected out of the sleeve 2, the fastening ring 18 becomes in contact with the C-ring 3 at the proximal portion thereof, and the arms 11 are be closed.

Referring back to FIG. 1, in the flexible sheath 1 at the distal end portion, the clip assemblies 10 aligned in line are mutually connected. More specifically, the tail portion 15 of the clip assembly 10 in a position closer to the distal end of the flexible sheath 1 (front side) and the arms 11 of the clip assembly 10 in a position closer to the proximal end of the flexible sheath 1 (rear side) define a connected portion. The claw portions 12 of the arms 11 of the clip assembly 10 in the rear side are closed to clutch the connecting loop 15a of the clip assembly 10 positioned in the front side.

The clipping instrument 100 includes an undetachable clip assembly 20, which is undetachably connected to the connecting loop 5. Unlike the clip assemblies 10, the undetachable clip assembly 20 does not include the fastening ring 18; however, the remaining configuration of the detachable clip assembly 20 is identical to the clip assembly 10. The undetachable clip assembly 20 is connected to the tail portion 15 of the clip assembly 10 being located at the rear end of the aligned clip assemblies 10, i.e., the arms 11 of the undetachable clip assembly 20 are closed to clutch the tail portion 15 of the clip assembly 10 at the rear end.

The arms 11 of the plurality of mutually connected clip assemblies 10 and the undetachable clip assembly 20 are closed inside the flexible sheath 1 so that the clipping instrument 100 is inserted into the forceps channel of the endoscope.

The clipping instrument 100 according to the embodiment of the present invention includes a frictional ring 31 and frictional cylinders 32, which serve to generate friction resistance with an inner surface of the flexible sheath 1 according to motions of the operation wire 4 being advanced and retracted.

The frictional ring 31 and the frictional cylinders 32 can be arranged in positions rather closer to the proximal end of the flexible sheath 1 than a position in which the flexible sheath 1 can be bended according to remote control by the operator through the operation unit.

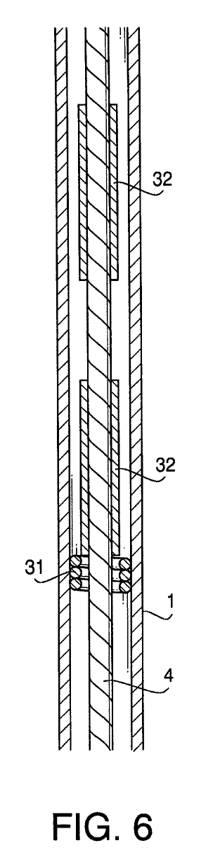

FIG. 6 is an enlarged cross-sectional side view of the flexible sheath with a frictional ring and frictional cylinders according to the embodiment of the present invention. According to the present embodiment, the frictional ring 31 is a resilient coil made of, for example, stainless steel wire being wound for a plurality of rounds in a predetermined diameter, and is pressed fit in the inner surface of the flexible sheath 1. The frictional cylinders 32 are, for example, pipes made of stainless steel and are fixed to and encircle the operation wire 4 with a predetermined interval provided therebetween.

The frictional ring 31 is configured to have an inner diameter smaller than the inner diameter of the flexible sheath 1 and an outer diameter slightly larger than the inner diameter of the flexible sheath 1. The frictional cylinders are configured to have outer diameters smaller than the inner diameter of the flexible sheath 1 and slightly larger (e.g., 0.05-0.1 mm) than the inner diameter of the frictional ring 31.

Therefore, when one of the frictional cylinders 32 passes through inside the frictional ring 31, the resilient frictional ring 31 is deformed to outstretch radially, a predetermined amount of friction resistance is generated between the frictional ring 31 and the frictional cylinder 32. Meanwhile, no friction resistance is generated when the frictional cylinder 32 is not included in the frictional ring 31.

As shown in FIG. 1, a number of the frictional cylinders 32 provided in the flexible sheath 1 is equated with a total number of the clip assemblies 10 and 20. Further, the intervals between the clip assemblies 10 and 20 are equated with the intervals between the frictional cylinders 32.

In addition, lengths of the frictional cylinders 32 and the positions on the operation wire 4 are determined in relation with the open/close operations of the clipping assemblies 10. More specifically, the lengths and the positions are configured such that the friction resistance starts to be generated when the operation wire 4 is advanced (i.e., from top to bottom in FIG. 1) and the distal end of the clip assembly 10 at the front becomes in contact with the distal end (i.e., the distal end of the sleeve 2) of the flexible sheath 1. The friction resistance is maintained while the clip assembly 10 passes through in the sleeve 2 to be projected out of the flexible sheath 1 and the arms 11 of the clip assembly become fully open. At the point when the arms 11 of the clip assembly 10 become fully open, the frictional cylinder 32 has passed through the frictional ring 31; therefore, the friction resistance is no longer generated.

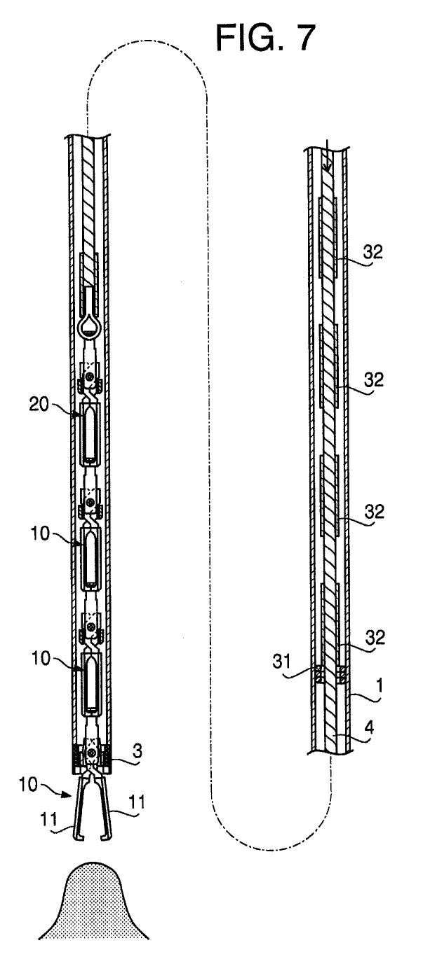

Operations of the clipping instrument 100 configured as above in use in the forceps channel will be described with reference to FIGS. 1 and 7-11. FIG. 7-11 are overall cross-sectional side views of the clipping instrument 100 in operation according to the embodiment of the present invention.

When the flexible sheath 1 is inserted into the forceps channel of the endoscope, as shown in FIG. 1, the clip assembly 10 at the front is entirely but scarcely included in the flexible sheath 1 so that the distal end of the clip assembly 10 is nearly aligned with the distal end of the flexible sheath 1 (i.e., the distal end of the sleeve 2). With the front clip assembly 10 in this position, the frictional cylinder 32 at the distal end is in contact with the proximal end of the frictional ring 31. Thus, the operator can sense the contact through the operation wire 4 and the operation unit and recognize the position of the front clip assembly 10 with hands.

When the flexible sheath 1 in the forceps channel is brought in the vicinity of the treatment area, as shown in FIG. 7, the operation wire 4 is advanced from the proximal end so that the front clip assembly 10 is projected out of the flexible sheath 1 and the arms 11 are opened gradually. Meanwhile, the frictional cylinder 32 at front passes through the frictional ring 32, and the friction resistance between generated therebetween is conveyed to the operator. Thus, the operator can recognize the arms 11 of the clip assembly 10 are not yet fully open.

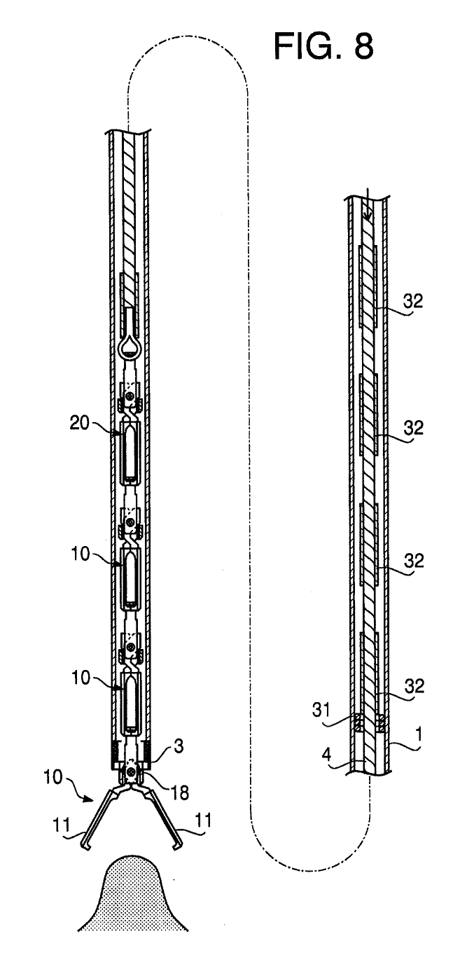

When the operation wire 4 is further advanced so that the front clip assembly 10 is projected out of the flexible sheath 1 and the fastening ring 18 passes through the C-ring 18, as shown in FIG. 8, arms 11 of the clip assembly 10 become fully opened by the effect of the fastening ring 18. Meanwhile, the frictional cylinder 32 at front has passed through the frictional ring 31; therefore, the operator senses abrupt cessation of the friction resistance and recognizes that the arms 11 of the front clip assembly 10 are fully open.

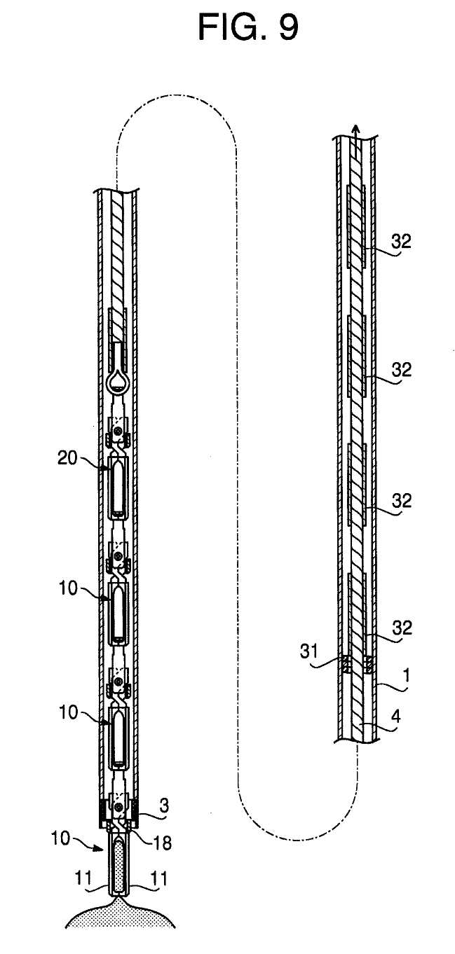

With the arms 11 opened, when the flexible sheath 1 is advanced, the arms 11 are closed to clutch the treatment area. Further, when the operation wire 4 is withdrawn toward the proximal end, as shown in FIG. 9, the arms 11 are substantially fully closed and the front clip assembly 10 is drawn back. In this state, the frictional cylinder 32 at front is drawn back in the frictional ring 31 again, and the operator senses the friction resistance. Thus, the operator can recognize the arms 11 of the front clip assembly 10 are closed.

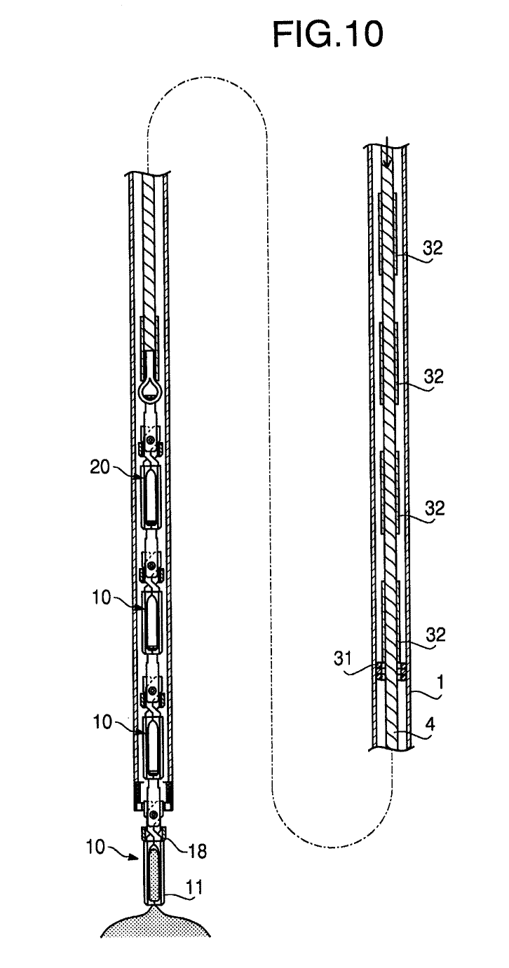

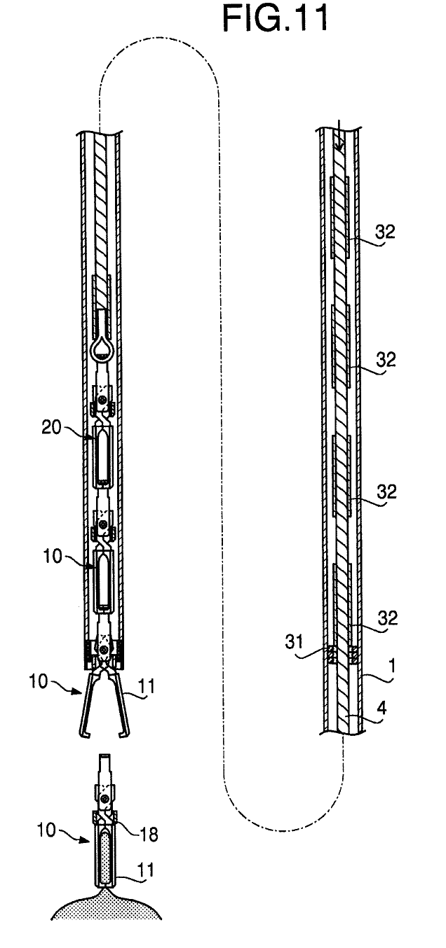

Thus, when the clipping operation is accomplished, the operation wire 4 can be again advanced from the proximal end, as shown in FIG. 10. The arms 11 of the front clip assembly 10 being closed to clutch the treatment area is advanced out of the flexible sheath 1. In this state, the frictional cylinder 32 at front is forwarded out of the frictional ring 31, and no friction resistance is caused to be sensed by the operator.

Next, as the operation wire 4 is further advanced and the distal end of the next clip assembly 10, which is second from the front end, comes to the position to substantially align with the distal end of the flexible sheath 1, the distal end of the frictional cylinder 32 at second from the front becomes in contact with the proximal end of the frictional ring 31. The contact can be conveyed to the operator so that the operator can recognize the position of the second clip assembly 10, which is entirely but scarcely included in the flexible sheath 1 so that the distal end of the clip assembly 10 is nearly aligned with the distal end of the flexible sheath 1.

When the operation wire 4 is further advanced, as shown in FIG. 11, the arms 11 of the second clip assembly 10 are opened, and the front clip assembly 10 is released from the connection. Thus, the front clip assembly 10 can remain clipping in the treatment area.

The above-described procedure can be repeatedly applied to the second and the following clip assemblies 10 as may be necessary.

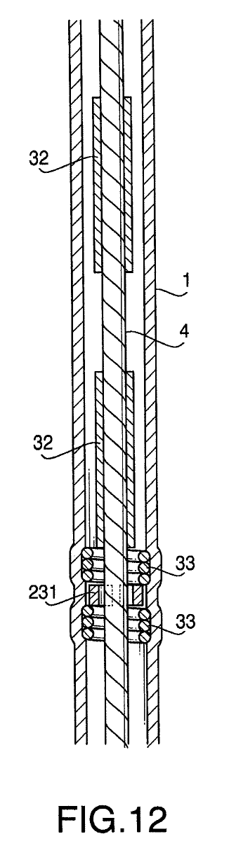

FIG. 12 is an enlarged view of a frictional ring 231 and frictional cylinders 32 according to a second embodiment of the present invention. The frictional ring 231 according to the present embodiment is formed to be a resilient C-ring with a slit. The frictional ring 231 is arranged to loosely fit inside the flexible sheath 1 and between a pair of retaining members 33. The retaining members are, for example, a pair of coils pressed fit inside the flexible sheath 1 and arranged to have a clearance therebetween. Thus, the frictional ring 231 of the present embodiment is held in the clearance by the retaining members 33. With this configuration, a clipping instrument to be operated similarly to the clipping instrument 100 of the first embodiment can be achieved.

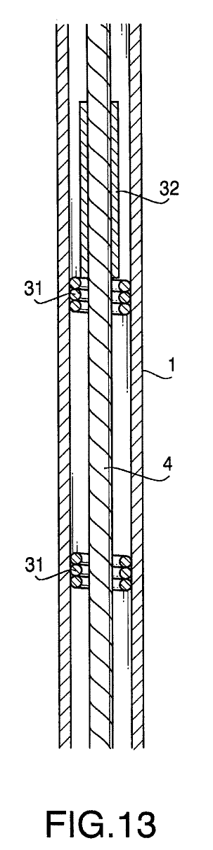

FIG. 13 is an enlarged view of frictional rings 31 and a frictional cylinder 32 according to a third embodiment of the present invention. In the present embodiment, solely one cylinder 32 is provided to be fixed to the operation wire 4 while the number of the frictional rings 31 is increased to be equated to the number of the clip assemblies 10 and 20. The intervals between the frictional rings are corresponded to the intervals between the clip assemblies 10 and 20. With this configuration, a clipping instrument to be operated similarly to the clipping instrument 100 of the first embodiment can be achieved.

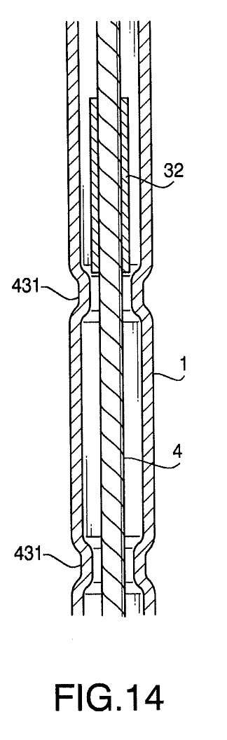

FIG. 14 is an enlarged view of frictional portions 431 and a frictional cylinder 32 according to a fourth embodiment of the present invention. According to the fourth embodiment, no independent frictional ring is provided; however, the flexible sheath 1 is plastically deformed to partially have smaller diameters to cause friction resistance with the frictional cylinders 32. With this configuration, a clipping instrument to be operated similarly to the clipping instrument 100 of the first embodiment can be achieved.

Although an example of carrying out the invention has been described above, the present invention is not limited to the above described embodiments. For example, a single clip assembly 10 may be provided instead of two or more clip assemblies 10 in the clipping instrument 100. For another example, the frictional cylinders 32 may be configured to be resilient while the frictional ring 31 is configured to be rigid.

The present disclosure relates to the subject matter contained in Japanese Patent Application No. P2007-152343, filed on Jun. 8, 2007, which is expressly incorporated herein by reference in its entirety.

What is claimed is:

1. A clipping instrument for an endoscope, comprising:
a flexible sheath to be inserted into an instrument channel of the endoscope;
an operation wire, which is configured to be forwarded and retracted in an axial direction of the flexible sheath;
at least one detachable clip assembly, including a pair of openable and closable clipping arms, the at least one detachable clip assembly arranged in vicinity of a distal end portion of the flexible sheath with the clipping arms closed, the clipping arms being configured to be projected forward to open outside the flexible sheath and to be closed to clip onto a treatment object outside the flexible sheath according to forwarding and retracting movements of the operation wire; and
frictional members, which are configured to generate frictional resistance between each other inside the flexible sheath,
wherein the frictional resistance is generated while the clipping arms of the clip assembly in the vicinity of the distal end portion of the flexible sheath are forwarded by the forwarding movement of the operation wire to project out of the flexible sheath to be fully opened.

2. The clipping instrument according to claim 1,
wherein the frictional members include at least one ring member, which is fixed to an inner surface of the flexible sheath, and at least one cylindrical member, which is fixed to and encircles the operation wire;
wherein the ring member is configured to have an inner diameter smaller than an inner diameter of the flexible sheath while the cylindrical member is configured to have an outer diameter smaller than the inner diameter of the flexible sheath and larger than the inner diameter of the ring member, and one of the ring member and the cylindrical member is resilient so that the resilient one of the frictional members deforms to allow the cylindrical member to pass through the inner diameter of the ring member according to the forwarding movement of the operation wire; and
wherein the frictional resistance is generated when the cylindrical member frictionally passes through the inner diameter of the ring member.

3. The clipping instrument according to claim 2, wherein the ring member is pressed fit inside the flexible sheath.

4. The clipping instrument according to claim 2,
wherein the ring member is arranged between a pair of retaining members, which are arranged to be pressed fit inside the flexible sheath along the axial direction of the flexible sheath with an clearance therebetween to hold the ring member.

5. The clipping instrument according to claim 2,
wherein the ring member is a resilient coil wound for a plurality of rounds in a predetermined diameter.

6. The clipping instrument according to claim 2, wherein the ring member is a resilient C-shaped ring.

7. The clipping instrument according to claim 2,
wherein the ring member is configured with a part of the flexible sheath being plastically deformed to have an inner diameter, which is smaller than the inner diameter of the other part of the flexible sheath.

8. The clipping instrument according to claim 1,
wherein a plurality of interconnected clipping assemblies, which are detachable from each other, are aligned in line inside the flexible sheath; and
wherein one of the plurality of clipping assemblies, which is arranged on a proximal end in the flexible sheath, is connected with the operation wire.

9. The clipping instrument according to claim 8,
wherein a number of the ring members provided in the flexible sheath corresponds to a number of the plurality of clipping assemblies; and
wherein intervals between the ring members correspond to pitches of the clipping assemblies aligned in line.

10. The clipping instrument according to claim 8,
wherein a number of the cylindrical members provided in the flexible sheath corresponds to a number of the plurality of clipping assemblies; and
wherein intervals between the cylindrical members correspond to pitches of the clipping assemblies aligned in line.
